(19) United States
(12) Patent Application Publication
(10) Pub. No.: US 2025/0327066 A1
(43) Pub. Date: Oct. 23, 2025

(54) IMPROVED METHOD FOR PREDICTING PROMOTER ACTIVITY

(71) Applicants: STICHTING HET NEDERLANDD KANKER INSTITUUT-ANTONI VAN LEEUWENHOEK ZIEKENHUIS, Amsterdam (NL); UMC UTRECHT HOLDING B.V., Utrecht (NL)

(72) Inventors: Jérémie Martin Breda, Amsterdam (NL); Bas Van Steensel, Amsterdam (NL); Lucia Barbadukka Martinez, Utrecht (NL); Jeroen De Ridder, Utrecht (NL); Noud Klaassen, Amsterdam (NL)

(21) Appl. No.: 19/119,269

(22) PCT Filed: Oct. 10, 2023
(86) PCT No.: PCT/NL2023/050526

§ 371 Date: Apr. 8, 2025

(30) Foreign Application Priority Data
Oct. 10, 2022 (NL) 2033267

(51) Int. Cl. C12N 15/10; G16B 20/20; G16B 40/20

(52) U.S. Cl. CPC C12N 15/1086; C12N 15/1065; C12N 15/1089; G16B 20/20; G16B 40/20

(57) ABSTRACT

This invention relates to a method of measuring gene promotor activity and a training data set consisting of promoter activity measurements for each of a plurality of DNA fragments. The invention also relates to a computer-implemented method for predicting gene promoter activity; and computer-readable storage medium or a computer program comprising computer-executable instructions which when executed by a computing system, are capable of causing the computing system to perform the method. The invention also relates to a computer-implemented method for training a deep learning (DL) model to predict gene promoter activity and the resulting trained model. Lastly, the invention also relates to various uses of the computer-implemented method for predicting gene promoter activity including predicting the effect of a carcinogenic mutation in a genome.

### FIELD OF THE INVENTION

The present invention relates to a method for measuring gene promoter activity. The invention also relates to a training data set optionally obtained by the method or comprising a measurement of promoter activity for each of a plurality of DNA fragments; and a computer-implemented method for predicting gene promoter activity wherein the model used in the method is trained on the training data set. The invention also relates to computer-readable storage medium or a computer program comprising computer-executable instructions to perform the computer-implemented method. The invention also relates to uses of the method, for example to predict a carcinogenic mutation. Lastly, the invention also relates to a method of training a deep learning model to predict gene promoter activity and the trained model.

### BACKGROUND

Gene expression is largely driven by regulatory DNA sequences that are interspersed around and within genes. Differences in the sequence of such regulatory elements between individual organisms or between cells within an organism, referred to as mutations or genomic variants, can cause changes in gene expression, and lead to phenotypic changes and/or disease. Such sequence differences are thought to account for many human disorders, including cancer. They also account for thousands of traits in livestock, plants, and other organisms. Being able to predict the effects of specific changes in regulatory sequences thus has many applications in medicine and biotechnology.

However, being able to predict gene expression from sequence is important but difficult. Currently no methods exist that reliably predict such effects based on DNA sequence alone. A particular challenge is that these effects on gene expression can be cell-type specific. Any algorithm that predicts such effects from DNA sequence should therefore be able to be trained in an efficient and practical way to make cell-type specific predictions. Current algorithms developed for this purpose are not sufficiently reliable, due to underdeveloped algorithms and suboptimal training data.

As such, there is a need for improved algorithmic gene expression prediction.

### SUMMARY OF THE INVENTION

One method to measure directly how DNA sequences control gene expression is a class of assays named Massively Parallel Reporter Assays (MPRA). In these assays, thousands or millions of DNA fragments are tested for their effects on gene expression. The method is applicable to any organism of which cells can be transfected or transduced with a library of DNA fragments, including humans, mammals, plants and single-cell organisms.

However, none of these MPRAs have the scale and accuracy to determine the effect of the hundreds of millions of sequence variants that are found across entire human (or other organism) populations, or in mutated genomes such as in cancer. Moreover, MPRA with hundreds of millions of DNA fragments are expensive, time consuming, and technically very challenging.

In a first aspect of the invention a method of measuring gene promoter activity, the method comprising:
a) preparing a focused library comprising a plurality of DNA fragments each comprising a promoter sequence;
b) inserting the plurality of DNA fragments each comprising a promoter sequence into a reporter system which outputs a measurement of the promoter activity of the promoter sequence in each DNA fragment; and
c) measuring the promoter activity for the promoter sequence for each of the plurality of DNA fragments.

In a further aspect the invention provides a training data set consisting of promoter activity measurements for each of a plurality of DNA fragments, wherein at least 60% of the DNA fragments comprise promoter sequences, optionally wherein:
a) the data for the data set is obtained by the method of any of claims 1 to 6; and/or
b) each promoter sequence is represented by a plurality of overlapping DNA fragments, each DNA fragment comprising a different part of the promoter sequence.

In a further aspect the invention provides a computer-implemented method for predicting gene promoter activity, the method comprising:
a) inputting to a trained model a sequence comprising a gene promoter;
b) based on the sequence of the gene promoter, outputting from the trained model a prediction of the gene promoter activity,
wherein the model is trained using the training data set described above.

In a further aspect the invention provides a computer-readable storage medium or a computer program comprising computer-executable instructions, which when executed by a computing system, are capable of causing the computing system to perform the computer-implemented method described above.

In a further aspect the invention provides a computer-implemented method for training a deep learning (DL) model to predict gene promoter activity, the method comprising:
a) inputting the training data set according to claim 7 into a DL model running on one or more processors coupled to memory,
b) training the DL model to relate a promoter sequence with an associated measurement of promoter activity.

This aspect may be combined with the first aspect: a method of measuring gene promoter activity. That is the method of the first aspect may be performed then the method of training a deep learning model to form one method.

In a further aspect the invention provides a trained model obtained by the method for training a deep learning (DL) model described above.

In further aspects the invention provides a method for predicting the effect of a carcinogenic mutation in a genome, comprising performing the computer-implemented method for predicting gene promoter activity; and uses of the computer-implemented method for predicting gene promoter activity in any of the following:
predicting the effect of a mutation in a plant genome;
predicting the effect of a mutation in a mammalian genome, preferably in a livestock animal, farm animal, pet or a human;
predicting the effect of a mutation in an insect genome;
predicting the effect of a mutation in a microorganism genome, preferably in a fungus, bacterium or protist;
predicting the effect of a mutation in the genome of an in vitro and/or ex vivo cultured cell and/or cultured cell population;
designing of therapeutic and/or diagnostic interventions for diseases and/or disorders, preferably for mammalian diseases and/or disorders, even more preferably for human diseases and/or disorders.

### DETAILED DESCRIPTION

### General Terms

### Genomic DNA/DNA Fragments/Promoter Sequences

Promoter sequences can be 100-1000 base pairs in length. Therefore, the promoter sequence in the DNA fragment may be an entire promoter sequence or part of a promoter sequence. Promoter sequences may include for example, enhancer sequences with promoter activity. Gene promoter and promoter are used interchangeably throughout the specification.

The DNA fragments/genomic DNA may comprise any DNA fragment, derived from any possible origin such as, but not limited to, animal DNA, e.g. mammalian DNA e.g. human DNA, bacterial DNA, e.g. yeast DNA, but also viral DNA (e.g. DNA viruses) and the like. Moreover, it is contemplated that the DNA fragments may be from DNA of in vitro and/or ex vivo cultured cells and the like. Other sources of DNA from which DNA fragments may be derived and that may be suitable for use in the method of the invention are known to a skilled person.

### Method of Measuring Gene Promoter Activity

By gene promoter activity is meant how active the promoter is at transcribing a sequence downstream from the promoter. This can be measured by measuring the level of a barcode transcribed by the promoter sequence. The barcode may be a unique barcode. That is, each promoter sequence may have a unique barcode downstream of it, the transcription of which is measured to ascertain the activity of the gene promoter. The presence of a functional promoter will drive transcription of the barcode sequence into barcoded mRNA. These barcodes may then be counted after reverse transcription, PCR amplification and high-throughput sequencing. The promoter sequence, and the barcode associated with the promoter sequence (when used) may be sequenced by any sequencing method known in the art. The method therefore may additionally include sequencing the promoter sequences for each of the plurality of DNA fragments.

The method of measuring gene promoter activity may be for example for assembling training data.

By focused library is meant the library does not include DNA fragments for the entire genome. Instead, the DNA fragments are only or mainly only those which comprise promoter sequences. That is, it is a promoter-enhanced library. It is a library enriched for promoter sequences. That is, promoter sequences have been selected from the wider genome. The focused library may comprise or consist of at least 60%, at least 70%, at least 80% or at least 90% or 100% DNA fragments comprising a promoter sequence. The focused library may also include a set of non-promoter sequences. These help as “negative controls” that allow the Deep Learning algorithm to learn better which sequences do not act as promoters. These negative controls come from the non-promoter regions of the genome. Measurement of no promoter activity from these negative controls may be carried out in the same way as for the promoter sequences, i.e. within a reporting system. Encompassed by promoter sequence are also enhancer sequences with promoter activity. These may comprise 5-20% of the focused library.

By hybridization capture is meant a technique using a bait to capture sequences of interest and pull them out of a general sample. Here, the method uses DNA sequences complementary to promoter sequences to capture these from the general pool of fragmented genomic DNA. A tag is present on the bait DNA sequences which allows the DNA sequences to be pulled from the general sample resulting in a purified, promoter-focused library. The tag may be for example biotin. Preparing a focused library may also be done by synthesizing DNA fragments comprising promoter sequences.

By fragmented genomic DNA is meant the DNA is broken into double-stranded fragments. Fragmentation may be by physical shearing or enzymatic fragmentation. The resulting fragments may be sieved for selected sizes of fragments.

Specific cell lines may be any of the following: K562 (blood), HepG2 (liver), HCT116 (colon), MCF7 (breast) and LNCaP (prostate). By using only promoter-focused libraries, due to the reduced number of fragments, and the resulting reduction in complexity of the reporting system, the method of measuring gene promoter activity may be carried out for each different specific cell line. This provides more accurate results than using measurements from a general cell line to infer promoter activity for a specific cell line. Examples of promoter activity specific to specific cell lines can be found below. Examples are:
DGKE (Diacylglycerol kinase epsilon) & SLC2A1 (solute carrier family 2 member 1), which exhibit higher expression in LNCaPs compared to all other cell lines of >3 and >7 fold respectively
RPL15 (Ribosomal protein L15) showing >6-fold expression in MCF7 compared to other cell lines
AHR (Aryl hydrocarbon receptor) shows a >3-fold increased expression in K562 compared to all other cell

By reporter system is meant a tool used in molecular biology to interrogate the activity of multiple genetic regulatory elements. Various reporting systems may be used to measure the activity of each promoter sequence.

One example of a reporter system is SuRE (Survey of Regulatory Elements) This is a method comprising one or more of the steps of:
a) randomly fragmenting genomic DNA;
b) subject the DNA fragments to size selection, for example to obtain 0.1-2 kb (or 100-2000 bp) long fragments (e.g. 0.5 kb, 1 kb, 1.5 kb), preferably 0.1-1 kb long fragments, e.g. 0.1 kb, 0.5 kb, 1 kb etc;
c) ligate the DNA fragments into a plasmid, upstream of a transcription unit that does not comprise a promoter and comprising a random 20-bp barcode near its ′5 end, thereby obtaining a library of plasmids comprising the fragments;
d) high-throughput, paired-end sequencing of the resulting library, providing means for associating each barcode with the genomic start and end positions and orientation of the corresponding fragment;
e) transiently transfect cultured cells with the library.

SuRE represents a comprehensive MPRA, which redundantly queries genomic sequences as a series of partially overlapping fragments [1,2]. The redundancy and large coverage is particularly suited to train computational models such as those described below, e.g. DCNN. Moreover, SuRE represents a more direct measurement of mutation impact [2], leading to improved results. Finally, while SuRE measures promoter activity of DNA fragments, enhancers also act as promoters in this assay. However, other reporting systems can also be used.

### Training Data

The inventors, by means of the present invention, demonstrate that by using informative and balanced data (but less data, e.g. compared to previous methods, overall) is highly suitable for predicting the effect of non-coding variants. Therefore, it is herein proposed that focused reporting systems, for example a focused library input into SuRE, can be highly suitable for training of the DCNN. Further benefits of focused and cleverly designed DNA fragment libraries, for example SuRE DNA fragment libraries, substantially reduce the costs and labor associated with constructing the libraries and with generation of the MPRA training data. Further the invention allows for generating much better data across a much wider diversity of cell types, leading to accurate cell-type-specific predictions of the effects of sequence variants on gene activity. Moreover, focused libraries yield data of higher quality, which further benefits the quality of the DCNN predictions.

The training data set consists of promoter activity measurements for each of a plurality of DNA fragments, wherein at least 60%, at least 70%, at least 80%, at least 90%, at least 95% of the DNA fragments comprise or consist of promoter sequences. That is, it is a training data set enriched for DNA fragments comprising or consisting of promoter sequences. As explained above, by promoter sequence includes enhancer sequences with promoter activity. The level of these may be for example 5-20% of the overall DNA fragments (i.e. sequences) in the training data set. The level of true promoter sequences may be for example 70%-90% of the DNA fragments in the training data set.

Also present in the training data set are negative controls consisting of DNA fragments (i.e. sequences) which do not comprise or consist of promoter sequences and therefore have no promoter activity. These may be 5-10% of the overall DNA fragments (i.e. sequences) in the training data set. For clarity, the DNA fragments in the training set are the sequences of the DNA fragments, each with an associated promoter activity measurement. The sequences used for training may comprise or consist of the promoter sequences.

DNA fragments may be fragments comprising between 0.03-5Kb, for example, 0.1 kb-2 kb. The term “kb” is well-known in the field for identifying the length of a DNA, or fragment thereof. The DNA fragment in the training data set may be between 0.1 kb-1 kb in length. DNA fragments of various lengths may be provided for training.

Each promoter sequence in the training data set may be represented by a plurality of overlapping DNA fragments, each DNA fragment comprising a different part of the promoter sequence. That is, every promoter is split into multiple different DNA fragments, each of these individual DNA fragments having a promoter activity measurement. In this way, motifs within the promoter sequence can be interrogated. For example, if an entire promoter sequence is split across fragments A and B, with fragments A and B partially overlapping, but A gives a much higher activity than B: then A must contain a sequence motif (missing from B) that gives the promoter its high signal; or B must contain a motif (missing from A) that reduces the activity of B. By applying this logic to very large numbers of fragments, a causality model can be learned.

For example, each entire promoter (i.e. each promoter sequence from 5′ start to 3′ end) in the genomic DNA may be represented by 100 or more overlapping DNA fragments. This can be assessed by sequencing of the sequences in the training data set prior to training.

### The Model

The DL architecture may comprise a deep neural network, preferably a convolutional neural network (CNN), more preferably a deep convolutional neural network (DCCN).

The DL architecture may comprise a kernel comprising at least one, more preferably at least two, even more preferably at least three layers of processing units.

In one non-limiting embodiment of the present invention, the model or computer-implemented method, takes input one-hot encoded DNA sequences of up to 2000 bp, preferably of 1500 bp, for example, up to 600 bp. For example, the method takes as input one-hot encoded DNA sequences of up to 600 bp overlapping either putative enhancers or promoters spanning a region from ±300 bp upstream to ±100 bp downstream of the TSS (Transcription Start Site).

The model may comprise three 1-dimensional convolutional layers, preferably followed by one dense layer. For example, the model may comprise a stem 1-dimensional convolutional layer followed by a tower of five dilated convolutional layers, and a last dense layer.

The convolutional layers may comprise respectively 128, 64, and 32 kernels of size 20, 15, and 15. One or more, preferably all, convolution layers preferably comprise a rectified linear unit activation function, and kernel regularization. Each convolution preferably is followed by a max. pooling, a batch normalization, and a dropout with a probability of between 0.01-1, preferably about 0.1, in every three layers. Subsequently, the model performs a 1-dimensional global average pooling followed by a dense layer with a linear activation function and no regularization. The model can, for example, be trained for up to 40 epochs (e.g. 5, 10, 15, 20, 25, 30, 35, and any number in between) with a learning rate of about 10\hat{ }-4. The model training and prediction may be done with Tensorflow 2.9.1 and Keras 2.9.0, but a skilled person may be aware of other suitable model training and/or prediction tools.

The computer-implemented method may comprise one or more cycles and wherein the one or more cycles comprise a first phase (training) and a second phase (predicting),
wherein the first phase comprises selecting sequence data as a reference set and applying the DL architecture to the reference set to compute one or more reference sequence data; and
wherein the second phase comprises selecting sequence data as a set and applying the DL architecture to the set to identify differences between the sequencing data compared to the reference sequence data.

In an embodiment there is provided for a computer-implemented method in accordance with the present invention, wherein the first phase comprises one or more iterations, wherein an iteration comprises the partly or fully repeating the first phase.

Also encompassed by the present invention is that the current computer-implemented method can be combined with other deep-learning architectures. In one embodiment there is provided for a computer-implemented method as described herein further comprising one or more attention layers. In one further embodiment, there is provided for a computer-implemented method as described herein further comprising one or more transformer models. In one non-limiting example, the one or more attention layers can be (re) used as transformer models in the present computer-implemented method. In another non-limiting example the one or more attention layers and/or transformer models can be (re) used in the current computer-implemented method as described herein. For example, the current computer-implemented method may be combined with any one or more attention layers and/or transformer models provided herein. It is preferred that the current computer-implemented method is combined with the deep-learning architecture comprising convolutional blocks followed by transformer blocks.

By relate is meant the model finds the relationship between the input and the output. That is, it associates sequence motifs with a measurement of promoter activity.

### The Trained Model

### Input

The input for the trained model is any DNA sequence. The DNA sequence may be a putative gene promoter. Alternatively the DNA sequence may be a known gene promoter with a mutation or which is otherwise a variant.

### Output

Once the DL model is trained, it will predict the transcription activity of any DNA sequence (up to a certain size) that it is given. For example, it can predict the activity of any naturally occurring promoter in the genome, but also the activity of any promoter that carries one or more sequence variants/mutations. By comparing the predicted activity of the mutated promoter with the predicted activity of the non-mutated promoter, one can thus infer what the predicted impact is of the mutation. This can be done for any variant or mutation.

### Uses of the Trained Model

It is contemplated that the computer-implemented method in accordance to the invention is suitable for the identification of the effect of genomic variants, e.g. naturally occurring sequence variants (in non-coding regions), for example, in a human population that may contribute to, for example the risk, e.g. risk of occurring, prognosis, progression, outcome and the like, certain diseases/disorders.

The genomic variant may comprise a single nucleotide polymorphism (SNP).

### Computer Program and Non-Transitory Media

By computer program is meant machine readable program instructions. These may be provided on a transitory medium such as a transmission medium or on a non-transitory medium such as a storage medium. Such machine-readable instructions (computer program code) may be implemented in a high level procedural or object oriented programming language. However, the program(s) may be implemented in assembly or machine language, if desired. In any case, the language may be a compiled or interpreted language, and combined with hardware implementations. Program instructions may be executed on a single processor or on two or more processors in a distributed manner.

Therefore also included are one or more non-transitory computer readable media storing machine-readable instructions which, when executed, cause one or more processors to perform the method of any of claims 8-9.

Further aspects of the invention are set out below as clauses and can be combined with any of the aspects described above.

Clause 1. A computer-implemented method for predicting gene expression of a DNA sequence, the method comprising:
a) providing sequencing data;
a) providing a deep-learning (DL) architecture running on one or more processors coupled to memory,
b) processing said sequencing data as input for the DL architecture;
c) obtaining a classification score output from the DL architecture for predicting gene expression for the DL architecture to identify one or more genomic variants.

Clause 2. The computer-implemented method according to clause 1, wherein sequencing data comprises one or more sequences and/or the gene expression of one or more sequences.

Clause 3. The computer-implemented method according to any one of the previous clauses, wherein the sequencing data is obtained by performing the steps of:
a) providing one or more DNA fragments;
b) contacting the DNA fragments with a barcode, thereby forming barcoded plasmids comprising the DNA fragments;
c) transfecting the barcoded plasmids obtained in step b) into one or more cells, thereby obtaining one or more cells comprising DNA comprising the DNA fragments comprising a barcode;
d) transcribing the DNA comprising the DNA fragments comprising a barcode;
e) sequencing the transcribed genomic fragments comprising a barcode to obtain sequencing data for processing as input for the DL architecture.

Clause 4. The computer-implemented method according to any one of the previous clauses, wherein the sequencing data is from one or more DNA fragments comprising a promoter sequence and/or enhancer sequence.

Clause 5. The computer-implemented method according to any one of the previous clauses, wherein the DL architecture comprises a kernel comprising at least one, more preferably at least two, even more preferably at least three layers of processing units.

Clause 6. The computer-implemented method according to any one of the previous clauses, wherein the DL architecture comprises a kernel comprising:
a first layer comprising a position weight matrix and/or position frequency matrix to detect a motif and generate a weight based on the motif;
a second layer comprising a processing unit to identify one or more transcription factor interaction;
a third layer comprising a processing unit to identify motif interaction, preferably between the motif of the first layer and of the second layer; and
wherein the kernel detects a motif in one or more layers, preferably in the first and second layer.

Clause 7. The computer-implemented method according to any one of the previous clauses, wherein the computer-implemented method comprises one or more cycles and wherein the one or more cycles comprise a first phase (training) and a second phase (predicting),
wherein the first phase comprises selecting sequence data as a reference set and applying the DL architecture to the reference set to compute one or more reference sequence data; and
wherein the second phase comprises selecting sequence data as a set and applying the DL architecture to the set to identify differences between the sequencing data compared to the reference sequence data.

Clause 8. The computer-implemented method according to clause 7, wherein the first phase comprises one or more iterations, wherein an iteration comprises the partly or fully repeating the first phase.

Clause 9. The computer-implemented method according to any one of the previous clauses, wherein the deep-learning architecture comprises a deep neural network, preferably a convolutional neural network (CNN), more preferably a deep convolutional neural network (DCCN) even more preferably a CNN-based genomic variant classifier.

Clause 10. The computer-implemented method according to any one of the previous clauses, wherein the classification score comprises a SuRE score.

Clause 11. The computer-implemented method according to any one of the previous clauses, wherein the DNA fragments are DNA fragments of a human genome.

Clause 12. The computer-implemented method according to any one of the previous clauses, wherein the DNA fragments are mutated DNA fragments or synthetic DNA fragments.

Clause 13. The computer-implemented method according to any one of the previous clauses, wherein the genomic variant comprises a single nucleotide polymorphism (SNP).

Clause 14. A method of training a deep learning architecture, preferably a convolutional neural network, even more preferably a convolutional neural network-based genomic variant classifier, to receive a sequence optionally comprising a variant and/or gene expression data and to generate a classification score for identifying a genomic variant in a sequence, the method comprising:
training the DL architecture, preferably by the convolutional neural network, more preferably the convolutional neural network-based genomic variant classifier, using as input one or more sequences, preferably wherein the input comprises one or more sequences and/or sequence expression data of a promoter sequence and/or enhancer sequence, paired with reference sequences and/or gene expression data,
whereby the DL architecture, preferably the convolutional neural network, more preferably the convolutional neural network-based genomic variant classifier, classifier after training is configured for receiving one or more sequences and/or gene expression data, preferably one or more sequences and/or gene expression data of a promoter sequence and/or enhancer sequence, and generating a classification score output for identifying a genomic variant.

Clause 15. A method for predicting a carcinogenic mutation in a genome, comprising performing the computer-implemented method according to any one of the previous clauses, wherein the sequencing data, preferably one or more sequences and/or the gene expression of one or more sequences comprises a sequence and/or gene expression of a sequence that is, or is suspected, of being carcinogenic.

Clause 16. A system comprising one or more processing units comprised in one or more processors, coupled to memory, the memory comprising computer instructions for implementing a DL architecture, preferably a CNN, more preferably a CNN-based genomic variant classifier, comprising:
an input unit that receives sequencing data;
a kernel comprising at least one, more preferably at least two, even more preferably at least three layers of processing units, preferably comprising:
a first layer comprising a position weight matrix and/or position frequency matrix to detect a motif and generate a weight based on the motif;
a second layer comprising a processing unit to identify one or more transcription factor interaction;
a third layer comprising a processing unit to identify motif interaction, preferably between the motif of the first layer and of the second layer;
wherein the kernel detects a motif in one or more layers, preferably in the first and second layer;
a DL architecture, preferably a CNN, more preferably a CNN-based genomic variant classifier,
trained on sequencing data, preferably wherein the input comprises sequencing data of one or more DNA fragment comprising a promoter sequence and/or enhancer sequence, paired with reference sequencing data,
that generates a classification score of the sequencing data received by the input unit, using the input comprising the sequencing data and further using reference sequencing data and one or more weights;
an output unit that provides the classification score for predicting gene expression generated by the DL architecture, preferably CNN, more preferably CNN-based genomic variant classifier.

Clause 17. Use of the computer-implemented method according to any one of the previous clauses in any one of:
predicting a mutation in a plant genome;
predicting a mutation in a mammalian genome, preferably in a livestock animal, farm animal, pet or a human;
predicting a mutation in an insect genome;
predicting a mutation in a microorganism genome, preferably in a fungus, bacterium or protist;
predicting a mutation in the genome of an in vitro and/or ex vivo cultured cell and/or cultured cell population;
designing of therapeutic and/or diagnostic interventions for diseases and/or disorders, preferably for mammalian diseases and/or disorders, even more preferably for human diseases and/or disorders.

Also encompassed by the present invention are one or more steps comprising:
selecting of the most information-rich sequences from the genome of interest:
   based on information theory;
   based on biological knowledge and databases;
tuning the level of redundancy between partially overlapping fragments
   library complexity
including other sequence variants, preferably sequence variants that may maximally “challenge” the deep learning model, such as:
   randomly mutated sequences;
   mutated sequences predicted to constrain the model;
   sequences from one or more, preferably more, species;
   completely synthetic sequences;
   sequences with poor predictive performance on undertrained prediction models.

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular forms “a,” “an” and “the” include plural referents unless the context clearly dictates otherwise. For example, a method for administrating a fragment according to the invention includes the administrating of a plurality of such fragments (e.g. 10's, 100's, 1000's, 10's of thousands, 100's of thousands, millions, or more fragments).

As used herein, the term “and/or” indicates that one or more of the stated cases may occur, alone or in combination with at least one of the stated cases, up to with all of the stated cases.

As used herein, the term “comprising” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, the term “exemplary” means “serving as an example, instance, or illustration,” and should not be construed as excluding other configurations disclosed herein.

All references cited herein are incorporated by reference in their entirety.

The preceding description is presented to enable the making and use of the technology disclosed. Various modifications to the disclosed implementations will be apparent, and the general principles defined herein may be applied to other implementations and applications without departing from the scope of the appended claims. Thus, the technology disclosed is not intended to be limited to the implementations shown.

### DESCRIPTION OF THE FIGURES

FIG. 1 shows: Outline of a genome-wide MPRA (here named “SuRE”) that is used to generate training data for the DCNN algorithm. Genomic DNA from one or more individuals (in this example 4) is randomly fragmented. The fragments are cloned in a reporter construct that contains a random barcode sequence, followed by an open reading frame (ORF, typically encoding a fluorescent protein) and a polyadenylation signal (PAS). Each fragment is paired with a unique random barcode. The resulting library is sequenced in order to identify the fragments and their linked barcode. Next, the library is transfected or transformed into cultured cells. If a probed fragment contains promoter activity, the barcode will be transcribed into mRNA; the amount of mRNA reflects the strength of the promoter. Counting of the barcodes in mRNA by high-throughput sequencing can thus determine the promoter activity (here named “SuRE score”) for millions of fragments. These data are then used as input for DCNN training.

FIG. 2 shows: Principle of DCNN learning of MPRA data (here obtained by the SuRE assay). The sequence of each DNA fragment (“one-hot” encoded, “Input column”) and together with its measured activity (SuRE score) used as input for a convolutional neural net that consists of multiple layers (in this example: three layers). The layers roughly correspond to the levels of gene regulation by transcription factors: the first layer identifies short sequence motifs that are bound by transcription factors (TFs); the second layer identifies interactions between TFs; the third and possible additional layers identify higher-order combinatorial logic of TFs (“sequence grammar”). By training the DCNN on data from thousands, millions or more fragments, a model can be constructed that can predict promoter activity from DNA sequence alone.

FIG. 3 shows: Example of measured (“true”) and DCNN-predicted (“pred”) promoter activity of DNA fragments that overlap known human promoters, in orientation-dependent manner (“+” and “−”). Measured and predicted data from human K562 cells are shown as a genome-browser view of a representative part of the human genome. Position of genes is indicated at the bottom.

FIG. 4 shows: Example of predictive power of DCNN predictions trained on SuRE data. Scatter plots show activity of individual genomic DNA fragments. Left panel: comparison of two replicate SuRE experiments (measured data), illustrating the maximum correlation that may be expected due to noise in the measurements. Right panel: comparison of measured SuRE scores (x-axis, “REAL”) to predicted SuRE scores (y-axis, “PREDICTED”) of fragments that were not included in the training. At this single-fragment level, the DCNN predicts roughly 50% of the variance. Training data were from human K562 cells.

FIG. 5 shows: When data of individual genomic DNA fragments are combined for each promoter (typically ˜200 fragments per promoter), accuracy of DCNN predictions strongly improves. Data visualisation is the same as FIG. 4, but now binned by promoter. Note that the DCNN now predicts as much as 87% of the genome-wide variance.

FIG. 6 shows: Example of DCNN prediction of the effect of human single-nucleotide polymorphisms (SNPs) on promoter activity. X-axis shows the measured effect of SNPs (log 2-scale) as difference between reference (REF) and alternative (SNP) allele, according to genome-wide SuRE measurements in human K562 cells (van Arensbergen et al, Nat Genet. 2019; 51:1160-1169). Y-axis shows predictions according to DCNN model. Only SNPs that overlap promoter regions and with a significant measured effect are shown.

FIG. 7 shows: Example of validation of DCNN-predicted effects by available experimental mutagenesis data. Top panel: measured effects of individual base substitutions in the F9 promoter, as measured in a reporter assay (data from Kircher et al., Nat Commun. 2019; 10:3583). Positive values: mutation leads to increased promoter activity; negative values: mutation leads to reduced promoter activity. Bottom panel: same, but predicted by DCNN model that was trained on SuRE dataset that did not include any of these mutations. Note the similarity between predicted and measured effects (particularly the patch around position 690), even though the measured effects seem more noisy.

FIG. 8 shows: Example illustrating that SuRE-data-trained DCNN can predict effects of clinically relevant mutations in a promoter. Figure depicts three representations of DCNN predictions of the effects of single-nucleotide variants (SNVs) in the promoter of the human TERT gene. Overexpression of this gene contributes to the formation of cancer in a variety of tissues. In about 25% of all tumors, this is caused by one of two promoter mutations (C250T and C228T). Top panel: predictions of SNV effects in a series of TERT promoter fragments with different start and end positions. The positions of C250 and C228 are marked by boxes; in most fragments these mutations cause increased predicted activity. Middle panel: merged data from all fragments in the top panel. Mutations that are predicted to cause increased promoter activity are marked in blue. Note that only C250T but not C250A or C250G are predicted to have this effect; similarly, C228T but not C228A or C228G is predicted to cause increased promoter activity. Strikingly, only C250T and C228T are found in human tumors. Thus, the SuRE-data-trained DCCN correctly predicted the two clinically most relevant mutations. Bottom panel: alternative representation of the predictions, with negative values indicating mutations causing increased promoter activity, and positive values indicating mutations causing decreased promoter activity.

FIG. 9 shows: Identification of putative transcription factors (TFs) responsible for altered promoter activity. Top and middle panel are same as in FIG. 11; bottom panel shows TF motifs that fit mutated sequence motifs. TFs of the ELF family are predicted to bind to the C250T and C228T mutations.

FIG. 10 shows: Illustration of the robustness of DCNN predictions with respect to the amount of training data. Top row shows measured effects of mutations as determined in a reporter assay by Kircher et al., Nat Commun. 2019; 10:3583. Second row shows predicted mutagenesis profile of a human promoter when a DCNN is trained on a large SuRE dataset with ˜10 million fragments that overlap all well-annotated human promoters. Rows 3-10 show the predictions with when an increasing proportion of fragments is left out in the training session. Up to 95% of the training data could be left out without major loss of predictive power, indicating that smaller training libraries may be used.

FIG. 11 shows: Evidence that focused MPRA library can be produced by capture from a pool of randomly sheared genomic DNA fragments. In this example, fragments overlapping ˜30,000 transcription start sites (TSS) were captured from human genomic DNA. A) Hundreds of fragments overlapping each TSS can be obtained, yielding a library of roughly 15 million fragments total. The number of fragments can be adjusted to obtain optimal complexity for generating training data. B) Evidence that the captured DNA fragments can be highly enriched for fragments that overlap intended regions of the genome, in this case the 30,000 TSS. C) distribution of captured DNA fragment sizes, illustrating that a wide range of sizes can be captured. The size range can be further tuned by adjusting the random fragmentation of the input genomic DNA. Focused libraries can be obtained accordingly from the genome of any species.

FIG. 12 shows: Evidence that CNN model can be successfully trained on MPRA data from a focused library. CNN predicts measured promoter activity equally well when trained on a full-genome MPRA library (left) or a promoter-focused library (right). Results obtained with K562 human leukemia cells.

FIG. 13 shows: Examples of the predictive power of CNN modes trained on focused-library MPRA data generated in four different human cell lines (LnCAP: prostate cancer; HCT116: colon cancer; HepG2: liver cancer; MCF7: breast cancer).

FIG. 14 shows: Evidence the CNN model trained on a focused library with promoter fragments can accurately predict effects of mutations. Top panel: CNN-predicted effects of single-nucleotide substitutions on transcriptional activity of the human TERT promoter. Bottom panel: effects of the same substitutions as measured by a multiplexed reporter assay. In each panel, the size of each letter indicates the strength of the effect of the substitution of the nucleotide at that position (average of all three possible subsitutions, e.g. A can be substituted by C, G or T) and the direction if the letter (up or down) indicates whether the substitution leads to an increase or decrease in activity. Note the strong similarity between the measured and predicted effects at the majority of the positions throughout the promoter.

### EXAMPLES

Aspects of the present invention will now be illustrated by way of example only and with reference to the following experimentation.

### Example 1: Generation of Genome-Wide Training Data by SuRE

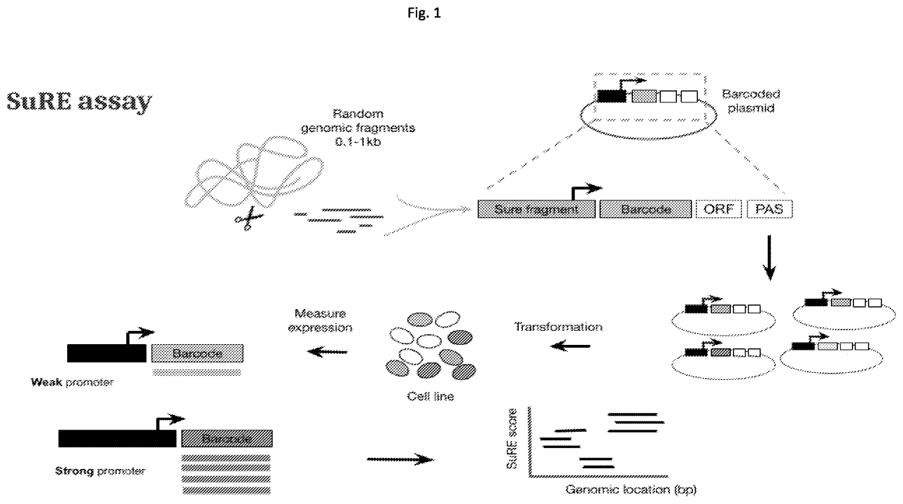

FIG. 1 shows the general pipeline for preparing the training data using the SuRE methodology. For genome-wide training libraries, this is done as previously published [1,2]. In brief, genomic DNA is randomly sheared or digested by one or more nucleases, and cloned into a plasmid vector pool that carries random barcodes. As a result, each genomic DNA fragment is linked to one barcode. The resulting library is sequenced to identify each fragment and its connected barcode. The library is then transfected into cultured cells. Fragments that harbor promoter activity will drive transcription of the corresponding barcode. Next, RNA is isolated from the cells, and sequenced by high-throughput sequencing. Barcodes in the RNA as thus counted. The abundance of each barcode is taken as a measure of the promoter activity of the corresponding fragment. Example 5-7 below describes how a library of specifically selected sequences can be used instead of a genome-wide library of fragments.

### Example 2: Preprocessing of the Training Data

Fragments were selected if they overlapped with a region of 300 bp upstream to 100 bp downstream of a list of selected TSS. The sequence of the fragments is then transformed into a one-hot matrix, where A is encoded as the vector [1, 0, 0, 0], C as [0, 1, 0, 0], G as [0, 0, 1, 0], and T as [0, 0, 0, 1]. All sequences are encoded into a matrix with a length of 600 bp; if the sequence is shorter than 600, it will be padded with the null vector [0, 0, 0, 0]. The amount of padding on the left and right of the sequence will be randomly distributed. Each fragment is associated with a score corresponding to the promoter activity; this score is normalized by the total read depth and log-transformed.

### Example 3: Building and Training the Computational Model

The method takes as input one-hot encoded DNA sequences of up to 600 bp overlapping either putative enhancers or promoters spanning a region from +300 bp upstream to +100 bp downstream of the TSS. The data was divided into 6 equal folds, with one serving as the test set, five as the training set, and the remaining one as the validation set. The validation and training sets were iteratively rotated to identify the best hyperparameters (number of layers, kernel size, filter size, learning rate, etc.) after training five different models. The architecture of the model comprises a stem 1-dimensional convolutional layer followed by a tower of five dilated convolutional layers, and a last dense layer. The convolutional layers include 125 kernels of size 7. All convolution layers comprise a rectified linear unit activation function, followed by a self-attention max pooling. Subsequently, the model performs a 1-dimensional global average pooling followed by a dense layer with a linear activation function and no regularization. The loss function employed to train the model was a Poisson function, accounting for the exponential distribution of the output values. The Adam algorithm (ADAMW (torch.optim.AdamW) from torch 1.12.1 was used) was chosen as the parameter optimizer. The model was trained for up to 20 epochs, but other epoch amounts are also possible. A gradual learning rate warm-up was applied for the initial training steps until reaching the appropriate learning rate of 10e-5 and gradient clipping of 0.2. Alternate padding was utilized to prevent systematically placing the sequence in the middle of the one-hot encoded matrix. The model training and prediction was performed with Tensorflow 2.9.1, Torch 1.12.1 and Keras 2.9.0.

### Results:

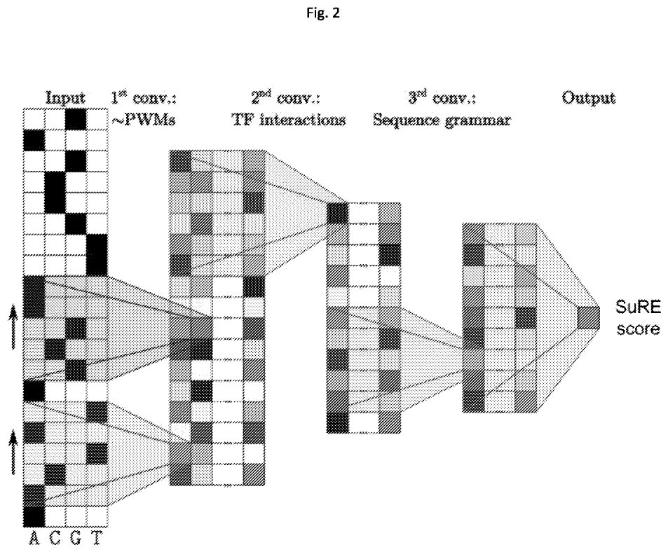

The results are shown in FIG. 2, which depicts the principle of DCNN learning of MPRA data (here obtained by the SuRE assay). The sequence of each DNA fragment (“one-hot” encoded, “Input column”) and together with its measured activity (SuRE score) used as input for a convolutional neural net that consists of multiple layers (in this figure: three layers, but other architecture such as those described in Example 3 can be used). The layers roughly correspond to the levels of gene regulation by transcription factors (TFs): the first layer identifies short sequence motifs that are bound by TFs; the second layer identifies interactions between TFs; the third and possible additional layers identify higher-order combinatorial logic of TFs (“sequence grammar”). By training the DCNN on data from thousands, millions or more fragments, a model can be constructed that can predict promoter activity from DNA sequence alone.

### Example 4: Testing the Model

To assess the model's performance on new data, we predicted the promoter activity of sequences that the model had never seen before. This was done in two ways:

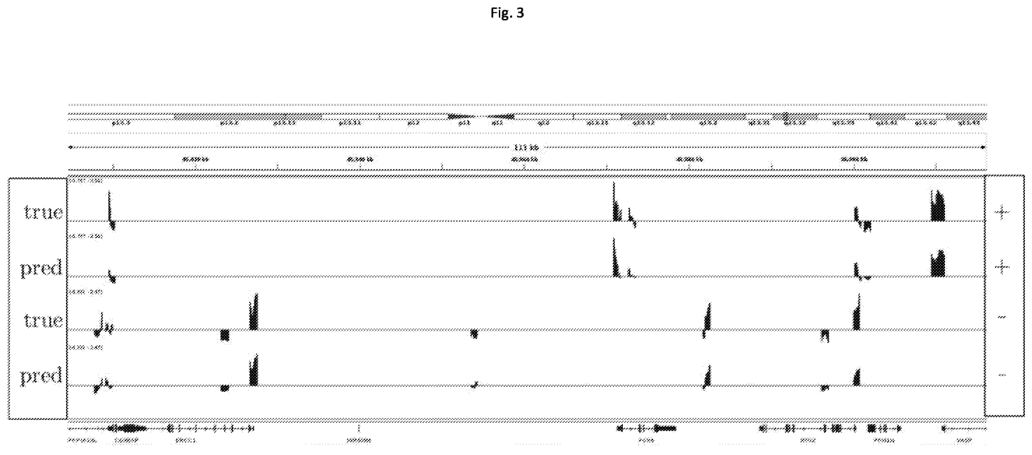

1. We routinely left out 10-20% of all promoters from the training data and then assessed how well the model predicted the activity of these promoters. These results are shown in FIGS. 3, 4 and 5.

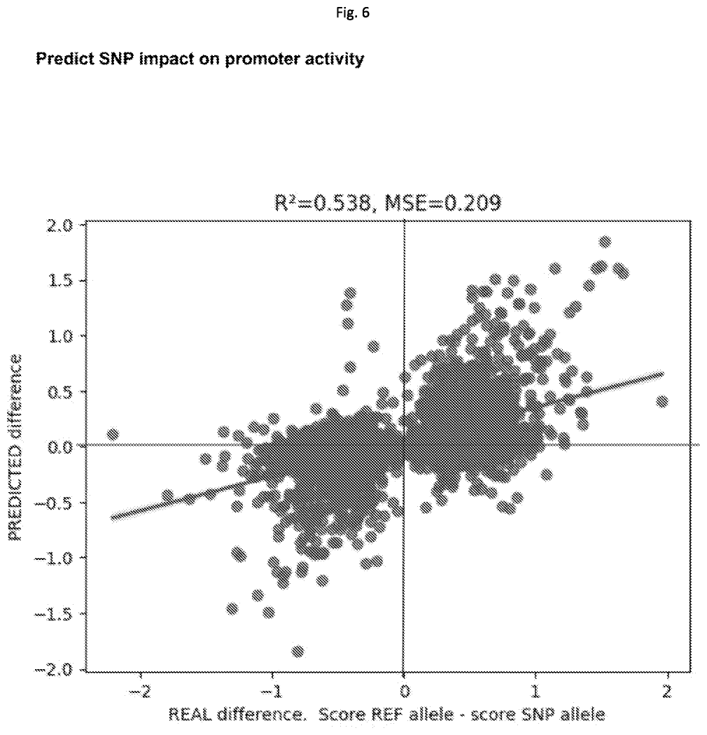

2. We tested whether the model could predict the effects of sequence variants as measured in dedicated MPRA assays in which many single-nucleotide substitutions were tested. The results are shown in FIGS. 6, 7, 8 and 10.

### Results:

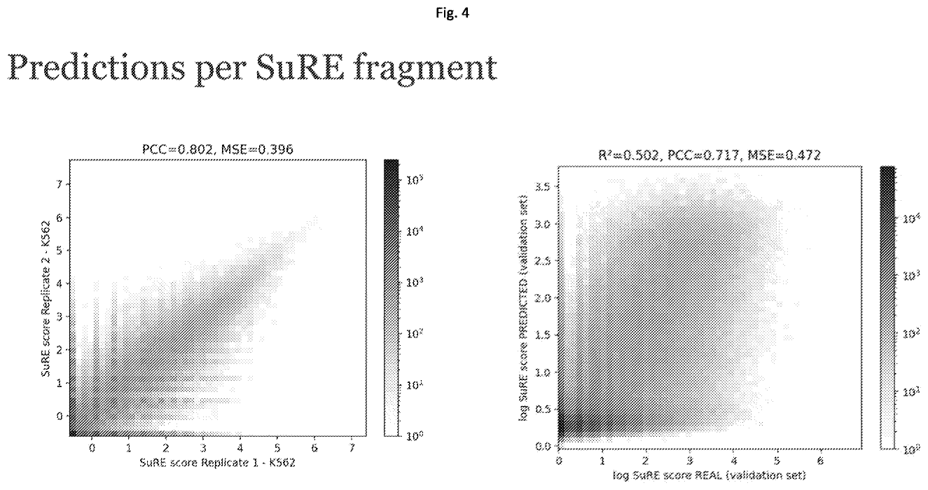

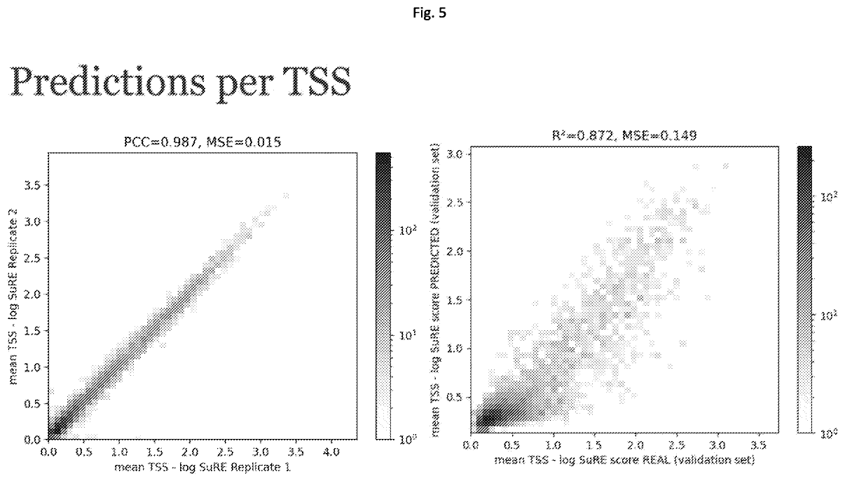

The results are shown in FIGS. 3, 4, 5, 6, 7, 8 and 10. FIG. 3 shows an example of a segment of the human genome, comparing measured to DCNN-predicted promoter activity in K562 cells, illustrating that the DCNN prediction is highly accurate. FIG. 4 shows how the activity of individual genomic DNA fragments is predicted by the model. FIG. 5 shows how the accuracy of DCNN predictions strongly improves when predictions for overlapping fragments are combined. FIG. 6 shows and example of DCNN prediction of the effect of human single-nucleotide polymorphisms

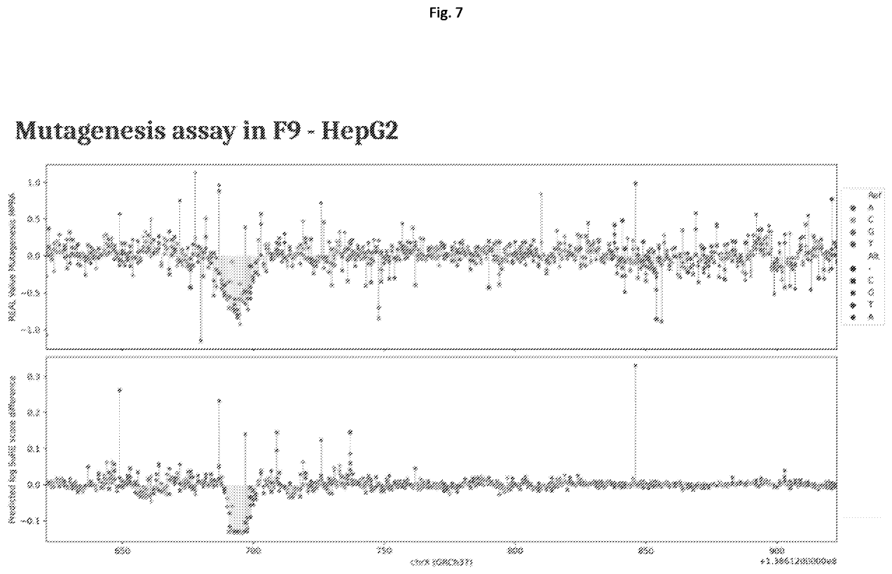

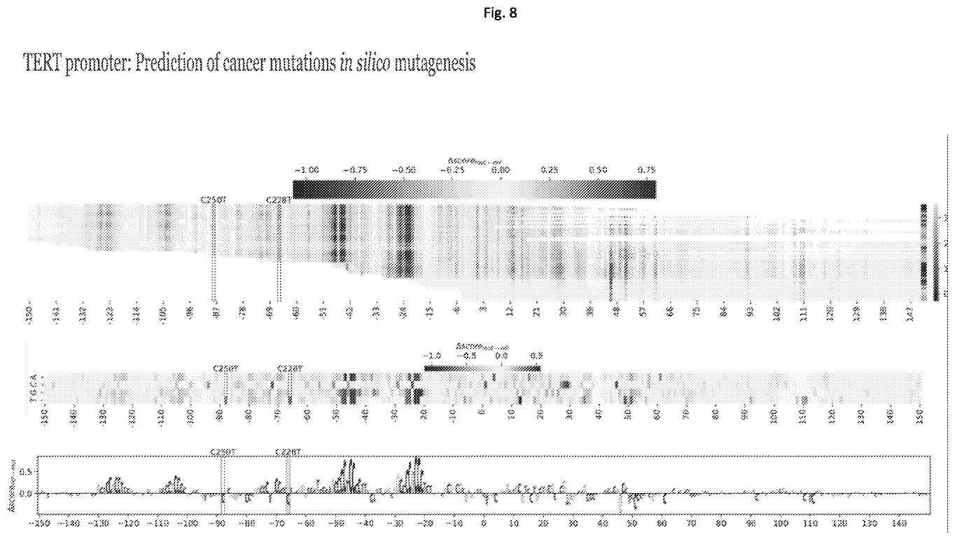

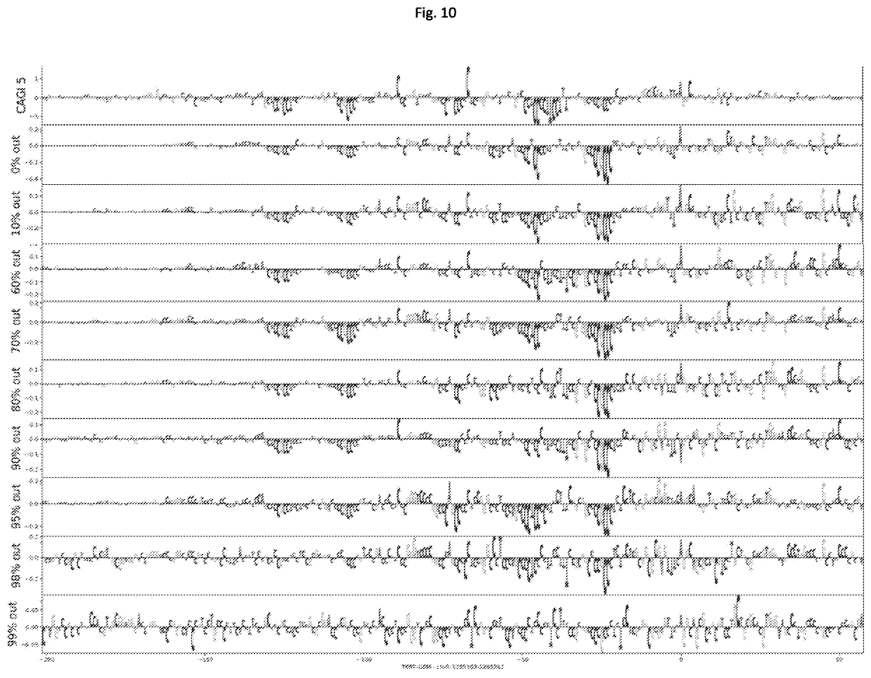

(SNPs) on promoter activity. FIG. 7 shows an example of validation of DCNN-predicted effects by available experimental mutagenesis data. FIG. 8 shows an example illustrating that SuRE-data-trained DCNN can predict effects of clinically relevant mutations in a promoter. FIG. 10 illustrates the robustness of DCNN predictions with respect to the amount of training data: even ˜95% of the training data can be left out without major impact on the accuracy of predictions of the effects of sequence variants. Together, these examples illustrate the power, accuracy and robustness of the models.

### Example 5: Extracting Biological Mechanisms from the Model

As illustrated in FIG. 2, the DCNN model implicitly models the activity of transcription factors (TFs). The identity of such TFs can be inferred from the model by in silico mutagenesis: for a given promoter, the predicted effect of all possible nucleotide substitutions in the promoter sequence can be calculated. This often reveals short patches of nucleotides that all can alter the activity. By comparing the sequence pattern of such patches to the binding specificity of known TFs, it is possible to identify the TFs that are candidates to be responsible for the regulation of the promoter.

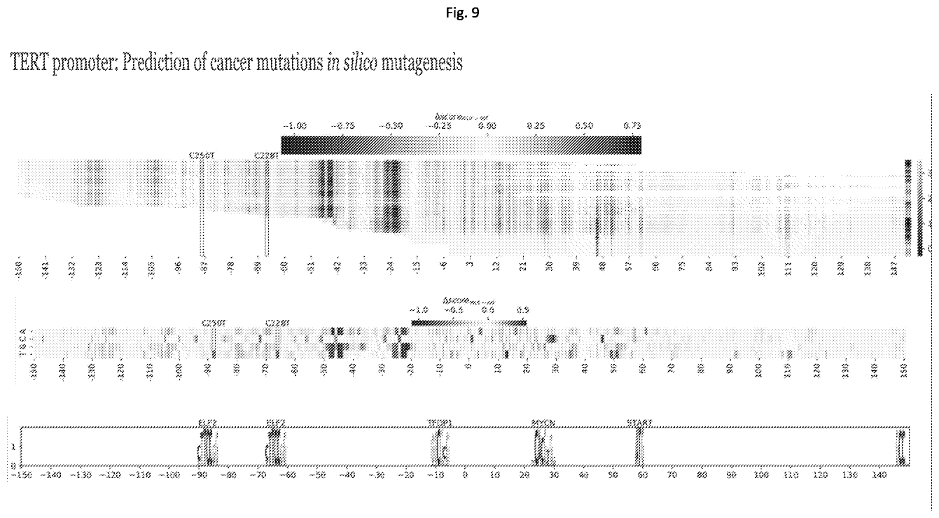

Results: An example is shown in FIG. 9, which depicts the identification of putative TFs responsible for altered promoter activity in the human TERT promoter in K562 cells.

### Example 6: Preparation of a Focused Training Library

Fragment selection and pre-processing. To generate a focused library we first isolated genomic DNA from a cultured human cell line (HG02601). The isolated DNA was then fragmented using dsDNA Fragmentase (NEB) and subsequently size-selected for fragments ranging from ˜200-400 bp. DNA was end-repaired using the End-IT DNA End-Repair Kit (Lucigen) and subsequently A-tailed using Klenow Fragment (3′->5′ Exo-; NEB). To be able to clone the DNA fragments into our vector after hybridization, we designed custom 31 bp dsDNA adapters containing a T-overhang for the 5′ and 3′ ends of the fragments. These adapters contain overlaps with the 3′ and 5′ ends of the linear barcoded vector respectively to allow Gibson assembly of the fragments after hybridization. Adapters were ligated to our fragments using TA-ligation.

Hybridization capture library design. To capture the promoter regions, we selected 30,607 well-annotated transcription start sites in the human genome and their-300 to +100 bp window and ordered a high stringency hybridization capture library for these custom regions consisting of 127,575 biotinylated DNA oligonucleotides (Twist Bioscience). To prevent nonspecific binding of probes to our fragments, we designed custom blockers (DNA oligonucleotides) complementary to the custom Gibson adapters.

Capture. Pre-processed fragments (as described above) were captured using the custom hybridization panel according to manufacturer protocols. Briefly, pre-processed fragments were hybridized to the hybridization library for 16 hours in presence of specific (custom designed) and nonspecific (Cot-1 DNA) blocker solution. Subsequently, hybridized fragments were enriched using streptavidin binding and amplified using custom primers using PCR for 9 cycles.

Library construction. Captured fragments were purified and cloned into our vector with Gibson assembly using the HiFi DNA assembly Master Mix (NEB). The Gibson assembly mix was then transformed into MegaX ultracompetent bacteria and this resulted in our final plasmid library.

To generate multiple complexities of libraries we generated multiple replicates consisting of ˜15M, ˜30M and ˜45M fragments.

### Results:

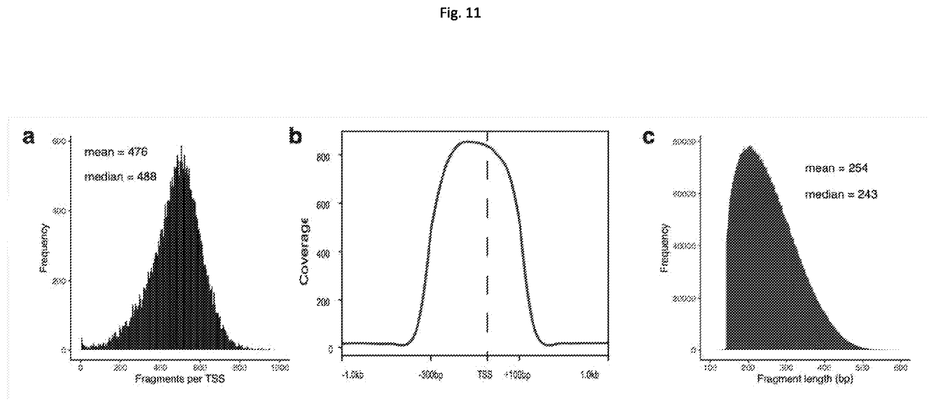

The results are shown in FIG. 11, which demonstrates that a focused MPRA library of high purity can be produced by capture from a pool of randomly sheared genomic DNA fragments.

### Example 7: Model Predicts Promoter Activity Using Less Data

The use of a focused library is much more economical and scalable than a full-genome training library. This saves labor and costs in terms of wet-lab experimental work, but also in terms of computing costs. Much less data needs to be processed—with consequent much reduced compute power requirements—to obtain powerful cell type specific models. Moreover, due to the simplified nature of the DL architecture (CNN-based), used in our models and in contrast to e.g. transformer-based architectures and/or much longer DNA sequence inputs, the required compute power to apply the model on e.g. all promoters in the (human) genome is also much reduced. This thus uniquely enables scanning of full genomes in an efficient manner and across many cell types.

### Results:

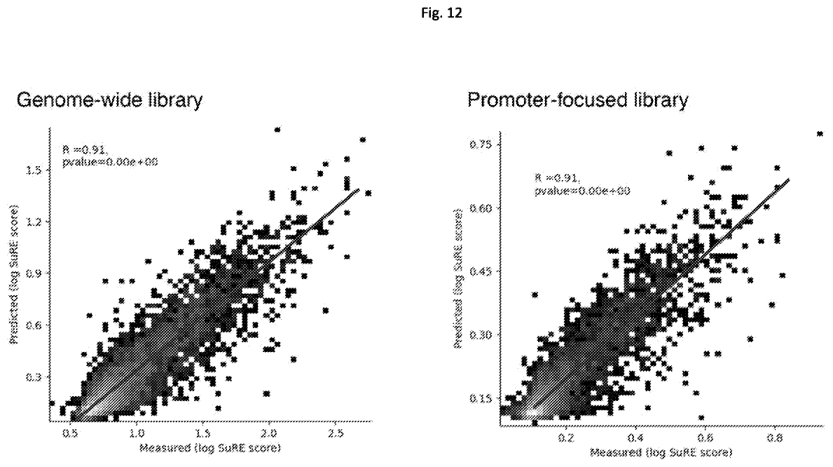

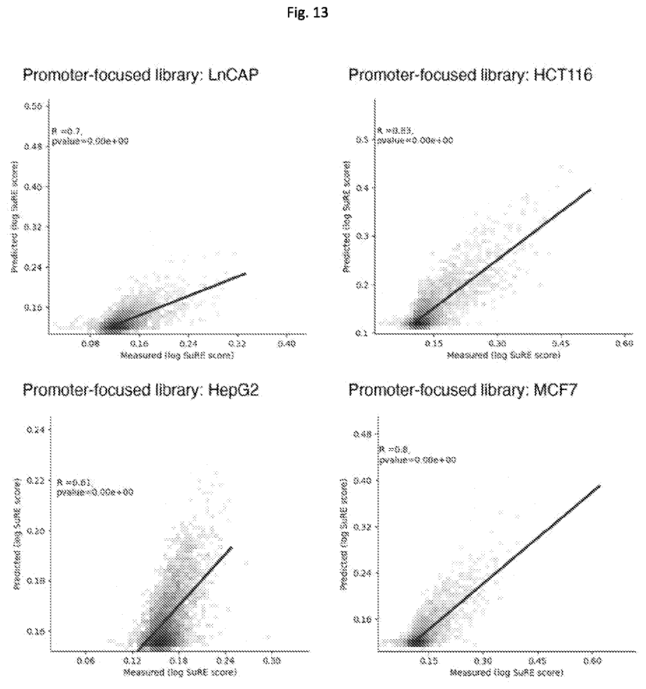

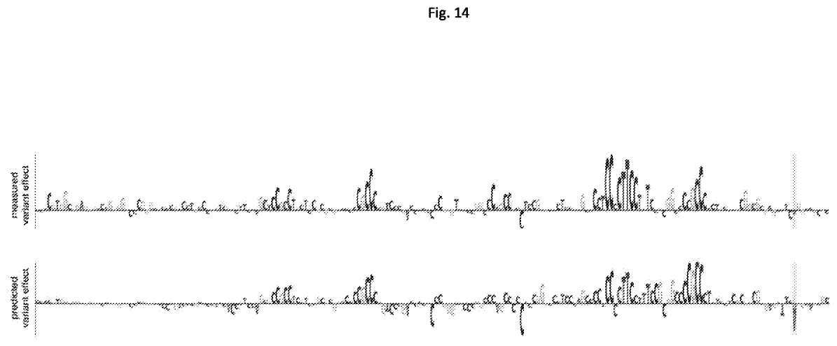

FIG. 12 demonstrates that data obtained with this promoter-focused library can yield a predictive DCNN model that is equally accurate as a model based on full-genome SuRE data. However, with the focused library only ˜10 million cells were required, while the data with full-genome libraries was obtained from about 1 billion cells total. This makes the procedure much more economical and scalable, without loss of quality. FIG. 13 provides proof-of-principle that the focused library can also be used to generate data and construct predictive models in various other cell types. FIG. 14 illustrates that the DCNN model obtained with the focused library can reliably predict the effects of sequence variants in a promoter.

In summary, despite using less data and ‘biasing’ the model with specific data selection, the model is as efficient as a model generated with the non-focused library. By using the focused data, not only is the model robust, the advantages are huge in that it provides a scalable approach across many cell types allowing cell-specific models to be made which can be vital in certain cell types.

### Example 8: The Trained Model can Predict Promoter Activity in Various Cancer Cell Lines

Four separate models were trained for MPRA data using a focused library on four different human cell lines: (LnCAP: prostate cancer; HCT116: colon cancer; HepG2: liver cancer; MCF7: breast cancer. Predicted versus measured activity scores demonstrate method efficacy across cell lines.

### Results:

The results are shown in FIG. 13 showing examples of the predictive power of CNN models trained on focused-library MPRA data generated in four different human cell lines (LnCAP: prostate cancer; HCT116: colon cancer; HepG2: liver cancer; MCF7: breast cancer).

FIG. 14 shows evidence that the CNN model trained on a focused library with promoter fragments can accurately predict effects of mutations, illustrated for the human TERT promoter.

In summary, FIG. 12 demonstrates that data obtained with the promoter-focused library can yield a predictive DCNN model that is equally accurate as a model based on full-genome SuRE data. However, with the focused library only ˜10 million cells were required, while the data with full-genome libraries was obtained from about 1 billion cells total. This makes the procedure much more economical and scalable.

FIG. 13 provides proof-of-principle that the focused library can also be used to generate data and construct predictive models in various other cell types. Lastly, FIG. 14 illustrates that the DCNN model obtained with the focused library can reliably predict the effects of sequence variants in a promoter.

### REFERENCES

[1] van Arensbergen J, FitzPatrick V D, de Haas M, Pagie L, Sluimer J, Bussemaker H J, van Steensel B. Genome-wide mapping of autonomous promoter activity in human cells. Nat Biotechnol. 2017 February; 35 (2): 145-153. doi: 10.1038/nbt.3754.
[2] van Arensbergen J, Pagie L, FitzPatrick V D, de Haas M, Baltissen M P, Comoglio F, van der Weide R H, Teunissen H, Vosa U, Franke L, de Wit E, Vermeulen M, Bussemaker H J, van Steensel B. High-throughput identification of human SNPs affecting regulatory element activity. Nature Genet. 2019 July; 51 (7): 1160-1169. doi: 10.1038/s41588-019-0455-2.

1. A method of measuring gene promoter activity, the method comprising:
a) preparing a focused library comprising a plurality of DNA fragments each comprising a promoter sequence;
b) inserting the plurality of DNA fragments each comprising a promoter sequence into a reporter system which outputs a measurement of the promoter activity of the promoter sequence in each DNA fragment; and
c) measuring the promoter activity for the promoter sequence for each of the plurality of DNA fragments.

2. The method of claim 1, wherein the promoter activity is measured in a specific cell line in the reporting system.

3. The method of claim 1 or 2, wherein the measurement of promoter activity is the level of a barcode transcribed by the promoter sequence.

4. The method of claim 3, comprising:
a) cloning the DNA fragments, each comprising a promoter sequence, into a reporter plasmid with a barcode, thereby forming barcoded plasmids comprising the DNA fragments;
b) transfecting or transfecting the barcoded plasmids obtained in step a) into one or more cells;
c) measuring the level of transcribed barcodes for each plasmid; and
d) sequencing the promoter sequences in each barcoded plasmid.

5. The method of any of the preceding claims, wherein preparing a focused library comprises:
a) performing hybridization capture of DNA fragments comprising promoter sequences from fragmented genomic DNA; or
b) synthesizing DNA fragments comprising promoter sequences.

6. The method of any of the preceding claims wherein each promoter sequence is represented by a plurality of overlapping DNA fragments, each DNA fragment comprising a different part of the promoter sequence.

7. A training data set consisting of promoter activity measurements for each of a plurality of DNA fragments, wherein at least 60% of the DNA fragments comprise promoter sequences, optionally wherein: a) the data for the data set is obtained by the method of any of claims 1 to 6; and/or
b) each promoter sequence is represented by a plurality of overlapping DNA fragments, each DNA fragment comprising a different part of the promoter sequence.

8. A computer-implemented method for predicting gene promoter activity, the method comprising:
a) inputting to a trained model a sequence comprising a gene promoter;
b) based on the sequence of the gene promoter, outputting from the trained model a prediction of the gene promoter activity,
wherein the model is trained using the training data set of claim 7,
optionally wherein the gene promoter comprises a variant,
further optionally wherein:
i) the variant is a single nucleotide polymorphism (SNP); and/or
ii) the variant is a potential pathogenic mutation, optionally wherein the method further comprises comparing the predicted gene promoter activity of the promoter comprising the potential pathogenic mutation with the predicted or observed gene promoter activity of the promoter without the potential pathogenic mutation.

9. The method of any of claims 1-8 wherein the genomic DNA is human genomic DNA or wherein the promoter sequence is a human promoter sequence.

10. A computer-implemented method for training a deep learning (DL) model to predict gene promoter activity, the method comprising:
a) inputting the training data set according to claim 7 into a DL model running on one or more processors coupled to memory,
b) training the DL model to relate a promoter sequence with an associated measurement of promoter activity.

11. The computer-implemented method according to claim 10, wherein the deep-learning model comprises a deep neural network, preferably a convolutional neural network (CNN), more preferably a deep convolutional neural network (DCCN).

12. A computer-readable storage medium or a computer program comprising computer-executable instructions, which when executed by a computing system, are capable of causing the computing system to perform the method of any one of claims 8 to 9.

13. A trained model obtained from the method of any one of claims 10 to 11.

14. A method for predicting the effect of a carcinogenic mutation in a genome, comprising performing the computer-implemented method according to any one of claims 8 to 9, wherein the one or more input sequences comprises a sequence that is, or is suspected, of being carcinogenic.

15. Use of the computer-implemented method according to any one of claims 8-9 in any one of:
predicting the effect of a mutation in a plant genome;
predicting the effect of a mutation in a mammalian genome, preferably in a livestock animal, farm animal, pet or a human;
predicting the effect of a mutation in an insect genome;
predicting the effect of a mutation in a microorganism genome, preferably in a fungus, bacterium or protist;
predicting the effect of a mutation in the genome of an in vitro and/or ex vivo cultured cell and/or cultured cell population;
designing of therapeutic and/or diagnostic interventions for diseases and/or disorders, preferably for mammalian diseases and/or disorders, even more preferably for human diseases and/or disorders.
